(19) United States
(12) Patent Application Publication
(10) Pub. No.: US 2009/0171147 A1
(43) Pub. Date: Jul. 2, 2009

(54) Surgical instrument

(76) Inventors: Woojin Lee, Hopkinton, MA (US); Andres Chamorro, Natick, MA (US); James C. Hollenbeck, Brighton, MA (US); Richard Ross, South Richford, VT (US)

Correspondence Address: David M. Driscoll, Esq., 1201 Canton Avenue, Milton, MA, 02186, US

(21) Appl. No.: 12/006,278

(22) Filed: Dec. 31, 2007

(51) Int. Cl. A61B 1/018; A61B 1/00

(52) U.S. Cl. 600/104; 600/137

(57) ABSTRACT

A medical instrument having a proximal control handle and a distal tool that are intercoupled by an elongated instrument shaft that is meant to pass internally of an anatomic body, proximal and distal movable members that respectively intercouple the proximal control handle and the distal tool with the instrument shaft, cable control means disposed between the movable members, an actuation member at the handle for controlling the distal tool through the movable members, and a coupler for selectively engaging or disengaging the shaft portion of the instrument from the handle portion. The handle has a distal receiver portion, and a shaft connector on said proximal motion member is selectively engageable with and releaseable from the receiver portion.

### TECHNICAL FIELD

The present invention relates in general to medical instruments, and more particularly to manually-operated surgical instruments that are intended for use in minimally invasive surgery or other forms of surgical or medical procedures or techniques. The instrument described herein is primarily for a laparoscopic procedure, however, it is to be understood that the instrument of the present invention can be used for a wide variety of other procedures, including intraluminal procedures.

### BACKGROUND OF THE INVENTION

Endoscopic and laparoscopic instruments currently available in the market are extremely difficult to learn to operate and use, mainly due to a lack of dexterity in their use. For instance, when using a typical laparoscopic instrument during surgery, the orientation of the tool of the instrument is solely dictated by the location of the target and the incision. These instruments generally function with a fulcrum effect using the patients own incision area as the fulcrum. As a result, common tasks such as suturing, knotting and fine dissection have become challenging to master. Various laparoscopic instruments have been developed over the years to overcome this deficiency, usually by providing an extra articulation often controlled by a separately disposed control member for added control. However, even so these instruments still do not provide enough dexterity to allow the surgeon to perform common tasks such as suturing, particularly at any arbitrarily selected orientation. Also, existing instruments of this type do not provide an effective way to hold the instrument in a particular position. Moreover, existing instruments require the use of both hands in order to effectively control the instrument.

An improved instrument is shown in U.S. Pat. No. 7,147,650 having enhanced dexterity and including, inter alia, a rotation feature with proximal and distal bendable members. Even though this instrument has improved features there remains the need for a more economically feasible instrument, and one in which the handle can be re-used while the tip of the instrument is disposable or reposable.

Accordingly, an object of the present invention is to provide an improved laparoscopic or endoscopic instrument in which a portion of the instrument is re-useable and a portion is disposable. In embodiments described herein the handle end of the instrument is re-useable and the distal portion or tip of the instrument is disposable. By being able to re-use the handle portion, the instrument is more economically feasible.

A further object of the present invention is to provide an improved laparoscopic or endoscopic surgical instrument that allows the surgeon to manipulate the tool end of the surgical instrument with greater dexterity.

Another object of the present invention is to provide an improved surgical or medical instrument that has a wide variety of applications, through incisions, through natural body orifices or intraluminally.

Another object of the present invention is to provide a locking feature that is an important adjunct to the other controls of the instrument enabling the surgeon to lock the instrument once in the desired position. This makes it easier for the surgeon to thereafter perform surgical procedures without having to, at the same time, hold the instrument in a particular bent configuration.

Still another object of the present invention is to provide an improved medical instrument that is characterized by the ability to lock the position of the instrument in a pre-selected position while enabling rotation of the tip of the instrument while locked.

Still another object of the present invention is to provide an improved medical instrument that can be effectively controlled with a single hand of the user.

### SUMMARY OF THE INVENTION

In accordance with the present invention there is provided a surgical instrument that includes: an instrument shaft having proximal and distal ends; a tool disposed from the distal end of the instrument shaft; a control handle coupled from the proximal end of the instrument shaft; a distal motion member for coupling the distal end of the instrument shaft to the tool; a proximal motion member for coupling the proximal end of the instrument shaft to the handle; actuation means extending between the distal and proximal motion members for coupling motion of the proximal motion member to the distal motion member for controlling the positioning of the tool; the handle having a distal receiver portion; a shaft connector on the proximal motion member selectively engageable with and releaseable from the receiver portion; and an actuation cable extending from the shaft connector to the tool for controlling the actuation of the tool.

In accordance with other aspects of the present invention the surgical instrument further includes a rotation means disposed adjacent the control handle and rotatable relative to the control handle for causing a corresponding rotation of the instrument shaft and tool; at least the proximal motion member comprises a proximal bendable member, the rotation means comprises a rotation knob that is adapted to rotate the tool about a distal tool roll axis and the rotation knob is disposed between the control handle and proximal bendable member; an actuation lever may be supported from the handle at a pivot point on the handle, a linkage mechanism controlled from the actuation lever and a cable engagement member controlled from the linkage mechanism for capturing a lug at the proximal end of the actuation cable for controlling the actuation cable and, in turn, the tool; the linkage mechanism may include a ratchet and pawl mechanism that provides successive lever positions for controlling the force applied at the tool and a plurality of connected links one of which includes a split link having a biasing spring therebetween; the cable engagement member may comprise a carriage that supports a gate that is movable transverse to the longitudinal axis of the carriage so as to capture the cable lug; a spring for biasing the gate and a cam block that is engageable with the gate to open the gate to enable the cable lug to be released; a locking mechanism for fixing the position of the tool at a selected position and having locked and unlocked states, the locking mechanism including a ball and socket arrangement disposed about the proximal motion member and a cinch member for locking the ball and socket arrangement; the socket member may comprises a split socket and the cinch member closes the split socket to lock the socket on the ball; a set of clamping blocks, the cable having distal of the lug a flange that is captured by the clamping blocks, the clamping blocks operated from a release member at the proximal end of the handle; including a sleeve member, a linkage member for controlling the transition of the sleeve member from the release member, the sleeve member controlling the clamping blocks to move toward and away from each other in providing the clamping action at the cable flange; including an actuation lever supported from the handle at a pivot point on the handle, the actuation cable having separable proximal and distal cable portions, the proximal cable portion controlled from the actuation lever, the distal cable portion selectively engageable or releaseable with respect to the proximal cable portion; including a cable locking mechanism for engaging the cable portions and a shaft locking mechanism for retaining the shaft connector; wherein the cable locking mechanism may include a sleeve and a release button mounted on the handle, and a connector at the distal end of the proximal cable portion that has multiple fingers for selective engagement with a lug on the distal cable portion so as to capture the cable lug, and wherein the shaft locking mechanism includes a gate and a release lever mounted on the handle, the gate for capturing a post on the distal cable portion; wherein the tool may include a collet, a removable tool member that is received in the collet and a set of jaws for holding the tool member; wherein the tool member may be a cautery tool and further including a voltage source at the handle for coupling energy to the actuation cable and an actuation lever for controlling the actuation cable which, in turn, controls the set of jaws for grasping the tool member; and wherein the tool may comprise a rotary cutter and further including a motor on the handle for controlling the rotary cutter via the actuation cable, and an actuation lever for controlling the actuation cable.

In accordance with the present invention there is also provided a medical instrument having a proximal control handle and a distal tool that are intercoupled by an elongated instrument shaft that is meant to pass internally of an anatomic body, proximal and distal movable members that respectively intercouple the proximal control handle and the distal tool with the instrument shaft, cable control means disposed between the movable members, an actuation member at the handle for controlling the distal tool through the movable members, a tool coupler for selectively engaging or disengaging the distal tool and a control member mounted at the handle for controlling the tool coupler.

In accordance with still other aspects of the present invention the medical instrument the tool coupler may includes a collet and a jaw member that transitions relative to the collet for receiving the distal tool and the collet may be attached to the proximal movable member, the proximal movable member comprises a proximal bendable member and the jaw member comprises a set of fingers extending from a base.

In still another embodiment there is provided a method of controlling a medical instrument that has a proximal end including a control handle and a distal end including a distal tool, the control handle and distal tool being intercoupled by an elongated instrument shaft and the tool actuated from a tool control cable that is operated from an actuation lever at the handle, the method including providing proximal and distal movable members that respectively intercouple the proximal control handle and the distal tool with the instrument shaft, the proximal and distal movable members being intercoupled so that a motion at the proximal movable member controls the distal movable member, and supporting the proximal movable member for removable interlock with a receiver portion at the handle.

In accord with other aspects the method may include dividing the tool control cable into separate cable segments and interlocking the separate cable segments so that the tool control cable is operable or manually controlling, from the proximal end of the instrument, the rotation of the distal tool about its longitudinal distal tool axis.

In still another embodiment the instrument has a proximal control handle and a distal tool that are intercoupled by an elongated instrument shaft, proximal and distal movable members that respectively intercouple the proximal control handle and the distal tool with the instrument shaft, means disposed between the movable members so that a motion at the proximal movable member controls the distal movable member and, in turn, the distal tool, means supported at the handle for controlling the distal tool including a tool control cable that extends between the proximal movable member and the distal tool and an actuation lever mounted at the handle, the handle having a distal receiver portion, and a shaft connector on the proximal movable member selectively engageable with and releaseable from the receiver portion.

In accordance with other aspects the tool control cable may include separate control cable segments that are adapted to have one of an engaged state and a disengaged state and a control member may be included at the control handle and manipulable by a user to control, via the proximal and distal movable members, the rotation of the distal tool about its distal tool axis.

### DESCRIPTION OF THE DRAWINGS

Numerous other advantages can be realized in accordance with the present invention by referring to the accompanying drawings, in which:

FIG. 1 is a perspective view of a first embodiment of a surgical instrument constructed in accordance with the present invention with a disposable shaft portion and a reusable handle portion, and illustrating the instrument in use;

FIG. 2 is a cross-sectional side view of the instrument of FIG. 1 with the actuation lever at rest and showing the jaws open;

FIG. 3 is an enlarged cross-sectional side view of the instrument of FIG. 1 and showing the instrument in use with the jaws at least partially closed;

FIG. 4 is an exploded fragmentary cross-sectional view illustrating the shaft removed from the handle;

FIG. 5 is a somewhat schematic cross-sectional detail view of the shaft locking means of the handle engaging the end of the shaft and with the cable engagement means at rest or unengaged;

FIG. 5A is a fragmentary detail view of the cable engagement means of FIG. 5 illustrating the cable lug being engaged;

FIG. 6 is a cross-sectional view similar to that shown in FIG. 5 but illustrating the cable lug being pulled proximally;

FIG. 7 is a fragmentary cross-sectional view showing the cable lug released and the shaft locking means disengaged from the shaft;

FIG. 8 is an exploded cross-sectional view similar to FIG. 7 but showing the instrument shaft removed from the handle;

FIG. 9 is an exploded perspective view of the shaft locking means and the cable engagement means disengaged;

FIG. 10 is a partially broken-away perspective view of the shaft and cable engagement means;

FIG. 11 is a partially broken-away perspective view of the cable engagement means by itself and in use;

FIG. 12 is a schematic side view of an alternate embodiment of the instrument adapted for use as a cauterization tool and employing a removable tip;

FIG. 12A is an end view of the tool clamping or holding means of FIG. 12;

FIG. 12B is a cross-sectional side view taken along line 12B-12B of FIG. 12A;

FIG. 13 is a fragmentary cross-sectional side view of the shaft and cable engagement means of FIG. 12;

FIG. 14 is a cross-sectional view taken along line 14-14 of FIG. 13 and illustrating the shaft locked;

FIG. 15 is an exploded cross-sectional view of the instrument of FIG. 12 showing the shaft removed from the handle;

FIG. 16 is a partially broken-away perspective view of the alternate instrument showing the cable engagement means by itself and in an engaged position;

FIG. 17 is an exploded perspective view similar to that shown in FIG. 16 but showing the cable engagement means released;

FIG. 18 is a schematic side view of a further alternate embodiment of the instrument adapted for use as a rotary cutting tool;

FIG. 18A is an end view of the tool clamping means of FIG. 18;

FIG. 18B is a cross-sectional view taken along line 18B-18B of FIG. 18A;

FIG. 18C is a fragmentary cross-sectional side view of the cable engagement means of FIG. 18;

FIG. 19 is a partially broken-away perspective view of the cable engagement means of FIG. 18 by itself and in an engaged position; and

FIG. 20 is an exploded perspective view showing the cable engagement means of FIG. 19 released.

### DETAILED DESCRIPTION

The present invention is illustrated in the drawings as a surgical instrument that has two portions such that a detachable instrument shaft portion may be disposable and a re-usable handle portion may be sterilized and reused numerous times. This allows for a higher quality instrument handle portion while keeping the overall price of the instrument reasonable.

The instrument of the present invention may be used to perform minimally invasive procedures. “Minimally invasive procedure,” refers herein to a surgical procedure in which a surgeon operates through a small cut or incision, the small incision being used to access the operative site. In one embodiment, the incision length ranges from 1 mm to 20 mm in diameter, preferably from 5 mm to 10 mm in diameter. This procedure contrasts those procedures requiring a large cut to access the operative site. Thus, the flexible instrument is preferably used for insertion through such small incisions and/or through a natural body lumen or cavity, so as to locate the instrument at an internal target site for a particular surgical or medical procedure. The introduction of the surgical instrument into the anatomy may also be by percutaneous or surgical access to a lumen, vessel or cavity, or by introduction through a natural orifice in the anatomy.

In addition to use in a laparoscopic procedure, the instrument of the present invention may be used in a variety of other medical or surgical procedures including, but not limited to, colonoscopic, upper GI, arthroscopic, sinus, thorasic, prostate, transvaginal, orthopedic and cardiac procedures. Depending upon the particular procedure, the instrument shaft may be rigid, semi-rigid or flexible.

Although reference is made herein to a “surgical instrument,” it is contemplated that the principles of this invention also apply to other medical instruments, not necessarily for surgery, and including, but not limited to, such other implements as catheters, as well as diagnostic and therapeutic instruments and implements.

There are a number of unique features embodied in the instrument that is described herein. For example, there is provided a locking mechanism that is constructed using a ball and socket arrangement disposed about the proximal motion member that follows the bending action and in which an annular cinch ring is used to retain the ball and socket arrangement in a fixed particular position, and thus also maintain the proximal and distal bendable members in a particular bent condition, or in other words locked in that position. The cinch ring includes a locking lever that is conveniently located adjacent to the instrument handle and that is easily manipulated to lock and unlock the cinch ring and, in turn, the position of the end effector. The cinch ring is also preferably rotatable to that the locking lever can be positioned conveniently or can be switched (rotated) between left and right handed users. This lock control allows the surgeon one less degree of freedom to concentrate on when performing certain tasks. By locking the bendable sections at a particular position, this enables the surgeon to be more hands-free for controlling other degrees of freedom of the instrument such as manipulation of the rotation knob to, in turn, control the orientation of the end effector.

A main feature of the present invention relates to the ability of the instrument to be partially disposable and partially re-useable. In that way the instrument cost can be substantially reduced as it is not necessary to replace the entire instrument for each procedure. In previous instrument constructions, the proximal bending member has been mounted directly to the rotation knob but now a connector and associated receiver allow the bending member to be removed from the rotation knob. In one embodiment a disconnect means is provided at the handle where the distal motion member, tool, instrument shaft and proximal motion member are separable from the handle of the instrument. This enables the distal components to be engageable and dis-engageable or releasable from the handle. The handle portion of the instrument is re-useable and thus the cost of that part of the instrument is essentially spread over several instrument uses.

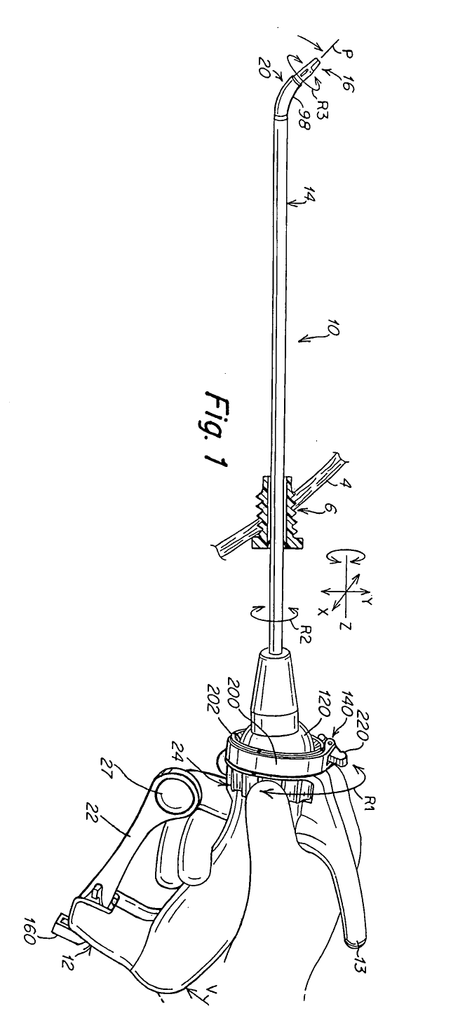

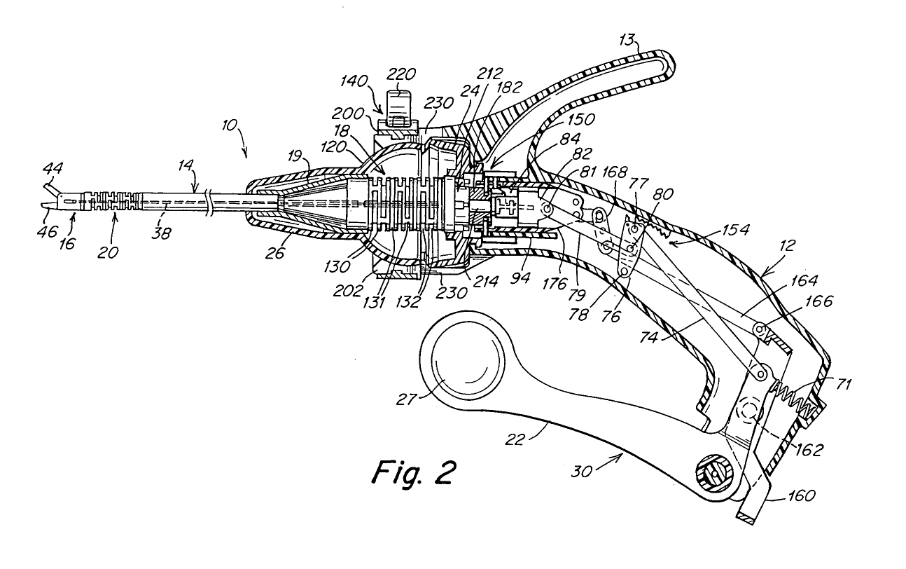

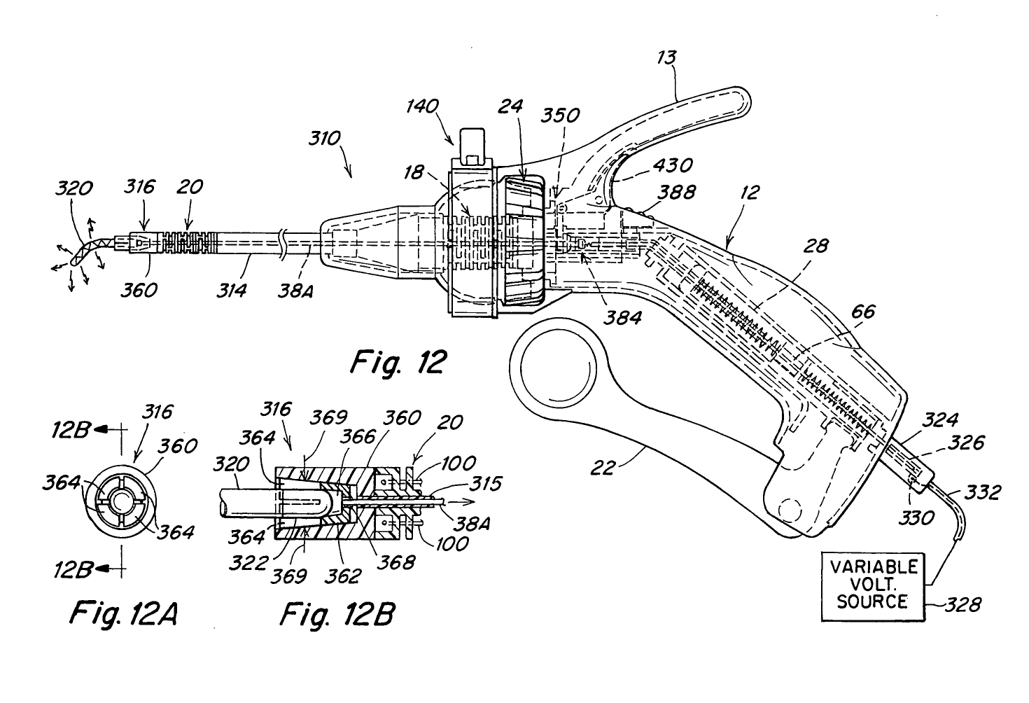

FIG. 1 is a perspective view of one embodiment of the surgical instrument 10 of the present invention. FIGS. 2-11 provide further details of this embodiment. FIGS. 12-17 illustrate a second embodiment of the present invention in which the instrument is adapted for use as a cauterization tool and employs a removable tip. FIGS. 18-20 illustrate a third embodiment of the present invention in which the instrument is adapted for use as a rotary cutting tool.

In the embodiment of FIG. 1 both the tool and handle motion members or bendable members are capable of bending in any direction. They are interconnected via cables (preferably four cables) in such a way that a bending action at the proximal member provides a related bending at the distal member. The proximal bending is controlled by a motion or deflection of the control handle by a user of the instrument. In other words the surgeon grasps the handle and once the instrument is in position any motion (deflection) at the handle immediately controls the proximal bendable member which, in turn, via cabling controls a corresponding bending or deflection at the distal bendable member. This action, in turn, controls the positioning of the distal tool.

The proximal member is preferably generally larger than the distal member so as to provide enhanced ergonomic control. In the illustrated embodiment the ratio of proximal to distal bendable member diameters may be on the order of three to one. In one version in accordance with the invention there may be provided a bending action in which the distal bendable member bends in the same direction as the proximal bendable member. In an alternate embodiment the bendable, turnable or flexible members may be arranged to bend in opposite directions by rotating the actuation cables through 180 degrees, or could be controlled to bend in virtually any other direction depending upon the relationship between the distal and proximal support points for the cables.

As has been noted, the amount of bending motion produced at the distal bending member is determined by the dimension of the proximal bendable member in comparison to that of the distal bendable member. In the embodiment described the proximal bendable member is generally larger than the distal bendable member, and as a result, the magnitude of the motion produced at the distal bendable member is greater than the magnitude of the motion at the proximal bendable member. The proximal bendable member can be bent in any direction (about 360 degrees) controlling the distal bendable member to bend in either the same or an opposite direction, but in the same plane at the same time. Also, as depicted in FIG. 1, the surgeon is able to bend and roll the instrument's tool about its longitudinal axis to any orientation simply by rolling the axial rotation knob 24 about a rotation direction indicated in FIG. 1 by the rotation arrow R1.

In this description reference is made to bendable members. These members may also be referred to as turnable members, bendable sections or flexible members. In the descriptions set out herein, terms such as “bendable section,” “bendable segment,” “bendable member,” or “turnable member” refer to an element of the instrument that is controllably bendable in comparison to an element that is pivoted at a joint. The term “movable member” is considered as generic to bendable sections and joints. The bendable elements of the present invention enable the fabrication of an instrument that can bend in any direction without any singularity and that is further characterized by a ready capability to bend in any direction, all preferably with a single unitary or uni-body structure. A definition of a “unitary’ or “uni-body” structure is—a structure that is constructed only of a single integral member and not one that is formed of multiple assembled or mated components—.

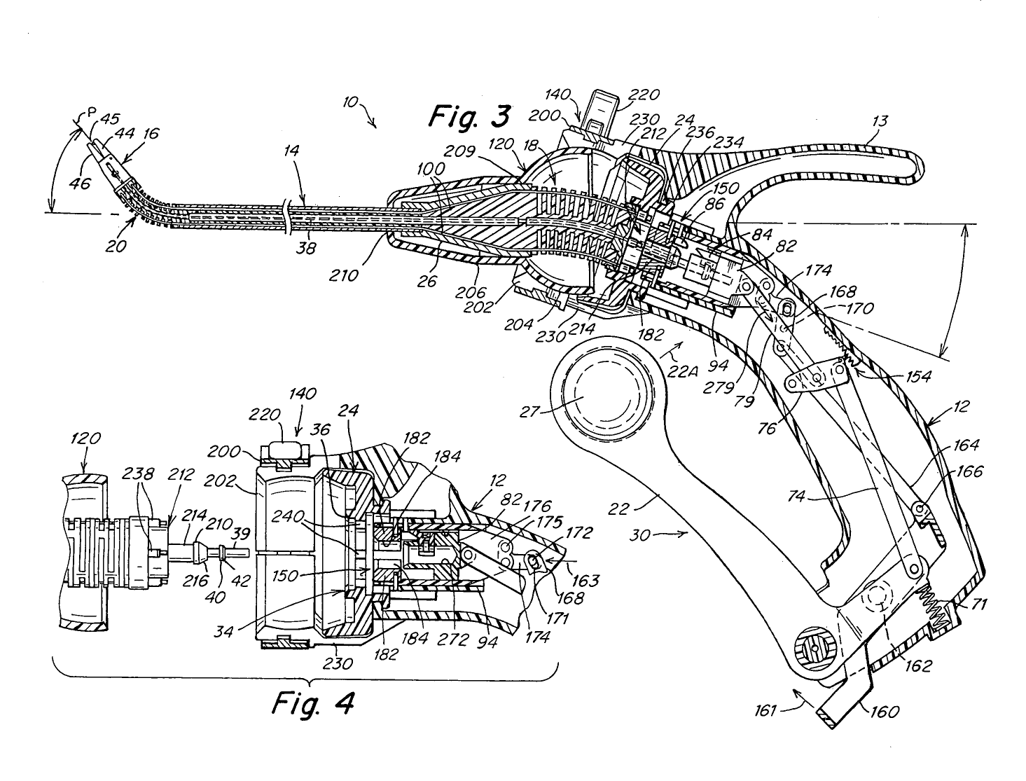

A definition of these bendable members is—an instrument element, formed either as a controlling means or a controlled means, and that is capable of being constrained by tension or compression forces to deviate from a straight line to a curved configuration without any sharp breaks or angularity—. Bendable members may be in the form of unitary structures, such as of the type shown herein in FIG. 3 for the proximal bendable member, may be constructed of engageable discs, or the like, may include bellows arrangements or may comprise a movable ring assembly. In FIG. 2 herein the unitary bendable structure includes a series of alternating flexible discs 130 that define therebetween slots 132. A “unitary” or “uni-body” structure may be defined as one that is constructed for use in a single piece and does not require assembly of parts. Connecting ribs 131 are illustrated as extending between adjacent discs 130. Both of the bendable members preferably have a rib pattern in which the ribs are disposed at a preferred 60 degree variance from one rib to an adjacent rib. For several forms of bendable members refer to co-pending applications Ser. No. 11/185,911 filed on Jul. 20, 2005; Ser. No. 11/505,003 filed on Aug. 16, 2006 and Ser. No. 11/523,103 filed on Sep. 19, 2006 all of which are hereby incorporated by reference herein in their entirety.

FIG. 1 shows one embodiment of the instrument of the present invention. Further details are illustrated in FIGS. 2 through 11. FIG. 1 depicts the surgical instrument 10 in a perspective view, as may occur during a surgical procedure. For example, the instrument may be used for laparoscopic surgery through the abdominal wall 4. For this purpose there is provided an insertion site at which there is disposed a cannula or trocar. The shaft 14 of the instrument 10 is adapted to pass through the cannula or trocar, that is schematically illustrated at 6, so as to dispose the distal end of the instrument at the operative site. The end effector 16 is depicted in FIG. 1. The embodiment of the instrument shown in FIG. 1 is typically used with a sheath 98 covering the distal member 20 to keep bodily fluids from entering the distal bending member 20.

A separate sheath (not shown) may be temporarily used to cover the entire distal bendable member and end effector. Such a sheath is only used for shipping the instrument and may be discarded once the instrument is in place on the handle. The sheath keeps the jaws in an open position, as illustrated in FIG. 2, and also keeps the distal bendable member in a substantially straight position. See related application Ser. No. 11/900,417 filed on Sep. 11, 2007, which is hereby incorporated by reference in its entirety, for further details of the temporary sheath construction. By doing that the actuation cable is maintained in a particular aligned position and ready for engagement with the handle portion of the instrument. Instead of using a pre-formed sheath one may alternatively use a biasing means in the instrument to maintain a predetermined position of the instrument cable, usually one in which the jaws are maintained open.

A rolling motion can be carried out with the instrument of the present invention. This can occur by virtue of the rotation of the rotation knob 24 relative to the handle 12 about a longitudinal shaft axis. This is represented in FIG. 1 by the rotation arrow R1. When the rotation knob 24 is rotated, in either direction, this causes a corresponding rotation of the instrument shaft 14. This is depicted in FIG. 1 by the rotational arrow R2. This same motion also causes a rotation of the distal bendable member and end effector 16 about an axis that corresponds to the instrument tip, depicted in FIG. 1 as about the longitudinal tip or tool axis P. In FIG. 1 refer to the rotational arrow R3 at the tip of the instrument.

Any rotation of the rotation knob 24 while the instrument is locked (or unlocked) maintains the instrument tip at the same angular position, but rotates the orientation of the tip (tool). For a further explanation of the tip rotational feature refer to co-pending application Ser. No. 11/302,654, filed on Dec. 14, 2005, particularly FIGS. 25-28, which is hereby incorporated by reference in its entirety.

The handle 12, via proximal bendable member 18, may be tilted at an angle to the instrument shaft longitudinal center axis. This tilting, deflecting or bending is in three dimensions. By means of the cabling this action causes a corresponding bend at the distal bendable member 20 to a position wherein the tip is directed along an axis and at a corresponding angle to the instrument shaft longitudinal center axis. The bending at the proximal bendable member 18 is controlled by the surgeon from the handle 12 by manipulating the handle in essentially any direction including in and out of the plane of the paper in FIG. 1. This manipulation directly controls the bending at the proximal bendable member. For further descriptions relating to the bending and locking features refer to co-pending application Ser. No. 11/528,134 filed on Sep. 27, 2006 and Ser. No. 11/649,352 filed on Jan. 2, 2007, both of which are hereby incorporated by reference in their entirety.

Thus, the control at the handle is used to bend the instrument at the proximal motion member to, in turn, control the positioning of the distal motion member and tool. The “position” of the tool is determined primarily by this bending or motion action and may be considered as the coordinate location at the distal end of the distal motion member. Actually, one may consider a coordinate axis at both the proximal and distal motion members as well as at the instrument tip. This positioning is in three dimensions. Of course, the instrument positioning is also controlled to a certain degree by the ability of the surgeon to pivot the instrument at the incision point or at the cannula or trocar. The “orientation” of the tool, on the other hand, relates to the rotational positioning of the tool, from the proximal rotation control member (knob 24), about the illustrated distal tip or tool axis P.

In the drawings a set of jaws is depicted, however, other tools or devices may be readily adapted for use with the instrument of the present invention. These include, but are not limited to, cameras, detectors, optics, scope, fluid delivery devices, syringes, etc. The tool may include a variety of articulated tools such as: jaws, scissors, graspers, needle holders, micro dissectors, staple appliers, tackers, suction irrigation tools and clip appliers. In addition, the tool may include a non-articulated tool such as: a cutting blade, probe, irrigator, catheter or suction orifice.

The surgical instrument of FIG. 1 shows one embodiment of a surgical instrument 10 according to the invention in use and may be inserted through a cannula at an insertion site through a patient's skin. Many of the components shown herein, such as the instrument shaft 14, end effector 16, distal bending member 20, and proximal bending member 18 may be similar to and interact in the same manner as the instrument components described in the co-pending U.S. application Ser. No. 11/185,911 filed on Jul. 20, 2005 and hereby incorporated by reference herein in its entirety. Some other components shown herein, particularly at the handle end of the instrument may be similar to components described in the co-pending U.S. application Ser. No. 11/528,134 filed on Sep. 27, 2006 and hereby incorporated by reference herein in its entirety. Also incorporated by reference in their entirety are U.S. application Ser. No. 10/822,081 filed on Apr. 12, 2004; U.S. application Ser. No. 11/242,642 filed on Oct. 3, 2005 and U.S. application Ser. No. 11/302,654 filed on Dec. 14, 2005, all commonly owned by the present assignee.

As illustrated in, for example, FIGS. 1-3, the control between the proximal bendable member 18 and distal bendable member 20 is provided by means of the bend control cables 100. In the illustrated embodiment four such control cables 100 may be provided in order to provide the desired all direction bending. However, in other embodiments of the present invention fewer or less numbers of bend control cables may be used. The bend control cables 100 extend through the instrument shaft 14 and through the proximal and distal bendable members. The bend control cables 100 may be constrained along substantially their entire length so as to facilitate both “pushing” and “pulling” action as discussed in further detail in the aforementioned co-pending application Ser. No. 11/649,352 filed on Jan. 2, 2007. The cables 100 are preferably constrained as they pass over the conical cable guide portion of the proximal bendable member, and through the proximal bendable member itself.

The locking means interacts with the ball and socket arrangement to lock and unlock the positioning of the cables which in turn control the angle of the proximal bending member and thus the angle of the distal bendable member and end effector. This lock control allows the surgeon one less degree of freedom to concentrate on when performing certain tasks. By locking the bendable sections at a particular position, this enables the surgeon to be more hands-free for controlling other degrees of freedom of the instrument such as manipulation of the rotation knob 24 and, in turn, orientation of the end effector.

The instrument shown in FIG. 1 is considered as of a pistol grip type. However, the principles of the present invention may also apply to other forms of handles such as a straight in-line handle. In FIG. 1 there is shown a jaw clamping or actuation means 30 that is comprised mainly of the lever 22 which may have a single finger hole in a gimbaled ball 27. The ball 27 is mounted at the free end of the lever 22. The surgeon uses the ball 27 for controlling the lever 22. In an alternate embodiment, the ball 27 is optional and in its place is a simple through or blind hole at the free end of the lever 22. There may also be provided a related release function controlled either directly by the lever 22 or a separate release button. The release function is used to release the tip of the instrument for interchange.

In the instrument that is illustrated the handle end of the instrument may be tipped or deflected in any direction as the proximal bendable member is constructed and arranged to preferably enable full 360 degree bending. This movement of the handle relative to the instrument shaft bends the instrument at the proximal bendable member 18. This action, in turn, via the bend control cables 100, bends the distal bendable member in the same direction. As mentioned before, opposite direction bending can be used by rotating or twisting the control cables through 180 degrees from one end to the other end thereof.

In the main embodiment described herein, the handle 12 is in the form of a pistol grip and includes a horn 13 to facilitate a comfortable interface between the action of the surgeon's hand and the instrument. The tool actuation lever 22 is shown in FIG. 1 pivotally attached at the base of the handle. The lever 22 actuates a linkage mechanism (see FIGS. 2 and 3) that controls the tool actuation cable 38. The cable 38 controls the opening and closing of the jaws, and different positions of the lever control the force applied at the jaws.

The instrument 10 has a handle portion 12 and a detachable shaft portion 14, as shown in FIG. 1. Many of the components of the instrument may be like that shown in Ser. No. 11/649,352 filed on Jan. 2, 2007, particularly as to the construction of the bendable members, instrument shaft, end effector, rotation member and locking mechanism. This includes means for enabling rotation of the shaft and proximal bendable member within bearings or bearing surfaces 208 and 210 (FIG. 3). The bearing 208 interfaces between the adaptor 26 and the ball 120, while the bearing surface 210 is between the neck portion 206 and the instrument shaft. The separate portions 12 and 14, or alternatively the assembled instrument, may be sealed in a sterile package or packages prior to storage or shipping.

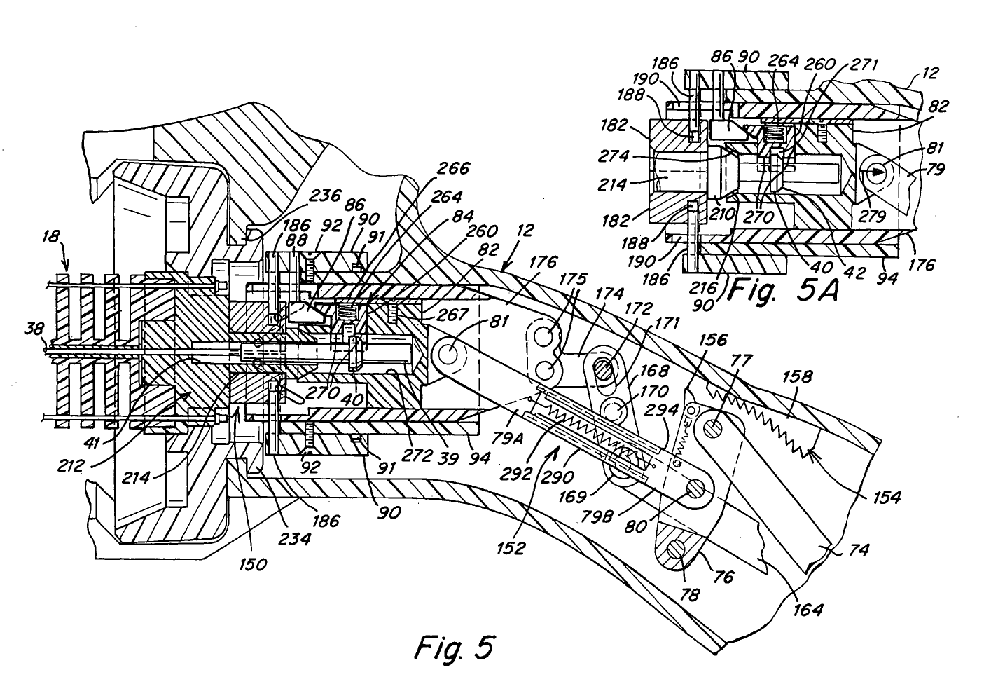

Reference is also now made to co-pending application Ser. No. 11/900,417 filed on Sep. 11, 2007 (which is hereby incorporated by reference in its entirety) for a description of a related instrument structure that includes a releasable shaft. The present invention is directed to further features particularly relating to the locking means for the shaft and for the cable lug. The locking means for the cable is actuation lever driven, and includes a spring loaded compensation means or member 152 (see FIG. 5) for constant jaw pressure applied to different thicknesses of tools or tissue, as well as a ratcheting means 154 to maintain the applied pressure. The members 152 and 154 are discussed in further detail hereinafter.

FIG. 2 shows the instrument in its rest position with the distal part of the instrument including the instrument shaft 14 engaged with the proximal part of the instrument including the control handle 12. FIG. 3, on the other hand shows, shows the instrument in a used position in which the lever 22 is at least partially depressed (moved toward the handle in the direction of the arrow 22A). In both of these views the distal part of the instrument is engaged with the proximal part of the instrument and the actuation cable is considered as interlocked or engaged so that operation of the lever 22 controls the movement of the actuation cable and in turn the actuation of the end effector 16. FIG. 4 is an exploded fragmentary cross-sectional view that depicts the distal part of the instrument (instrument shaft portion) having been removed from the control handle (instrument handle portion).

As shown in FIGS. 4 and 8, the shaft portion 14 can be easily separated from the handle portion 12 by releasing the cinch ring 200. For further details of the shaft portion release refer to co-pending application Ser. No. 11.900,417 filed on Sep. 11, 2007. The shaft portion 14 includes a shaft connector 212 (see FIG. 4). The shaft portion 14 is captured at the proximal flange 210 in the shaft receiver portion 34 of the rotation knob 24. Clamping blocks 182 capture the proximal flange 210. The shaft connector 212 is locked linearly but the shaft locking means or member 150 allows rotation of the shaft portion relative to the handle portion. The cable lug 40 is captured by means of the engagement thereof with the cable engagement means 84.

The instrument includes an angle locking means 140 as shown in FIGS. 1-4. This angle locking means includes a split hub 202 which is constructed and arranged to allow the ball 120, and the entire distal shaft portion, to be pulled out of the split hub 202. The cinch ring 200 is used to lock and unlock the split hub 202, as described in more detail later, and as further described in co-pending application Ser. No. 11/900,417 filed on Sep. 11, 2007.

The split hub 202 includes portions or petals that each preferably have a tapered face so as to function as a ramp to force the petals apart when the ball 120 is pushed proximally against them during an insertion of the shaft portion into the handle portion. These inward faces or edges of the portions are beveled or tapered to allow easier passage of the ball. The split hub 202 is supported from the handle by means of struts 230 which are thinned so as to function as flexible living hinges to thus allow more ready expansion of the hub petals. This structure assists in the engagement and disengagement between the shaft portion and handle portion.

The cinch ring 200 may have two flanges that ride in respective circumferential grooves that are disposed on the outer surface of the split hub 202. This interface captures the cinch ring while allowing the split hub to be separated linearly. The cinch ring 200 is basically controlled from the angle locking member or means 140. The angle locking member 140 is pivotally attached with the cinch ring 200. The angle locking member 140 is comprised primarily of the release/lock lever 220 which controls the length or outer circumference of the cinch ring 200. The angle locking member 140 is constructed and arranged to allow the cinch ring 200 to, not only be loosened enough to adjust the angle of the shaft relative to the handle, but to also expand to a size that is sufficient to allow enough expansion of the split hub portions to thus allow the ball 120 (and the entire distal shaft portion) to be removed or inserted in the split hub 202. This enables the shaft portion to be readily dis-engaged from the handle portion. For other details of the cinch ring construction refer to co-pending application Ser. Nos. 11/649,352 filed on Jan. 2, 2007 and Ser. No. 11/900,417 filed on Sep. 11, 2007.

The cinch ring 200 is operated by means of the over-center locking lever 220 that is connected to ends of the cinch ring 200 by means of the respective pins. The cinch ring 200 is free to rotate around the split hub 202 when lever 220 is released. This allows for left or right handed operation of the instrument. When the locking lever 220 is moved to its locked position this compresses the cinch ring 200 closing the hub against the spherical outer surface 204 of the ball member 120. This locks the handle against the ball member 120 holding the ball member in whatever position it is in when the locking occurs. By holding the ball member in a fixed position this, likewise, holds the proximal bendable member in a particular position and fixed in that position. This, in turn, maintains the distal bendable member and tool at a fixed position, but the instrument orientation can be controlled via the control of the rotation knob which controls the orientation of the instrument tip by enabling rotation of the distal bendable member and tool about the tip axis P (see FIG. 3).

Another feature of the instrument shown in the first embodiment is the use of a separate shaft release lever 160 shown in FIGS. 2 and 3. The lever 160 operates a linkage mechanism that, in turn, controls the shaft locking member 150. A sleeve 176 is controlled from the linkage mechanism and controls the opening and closing of clamping blocks 182. These blocks 182 capture the post 214 and the entire shaft portion. In an alternate embodiment the clamping blocks may capture the cable in a different way such as by having a projection on each block engage a slot or hole in the cable.

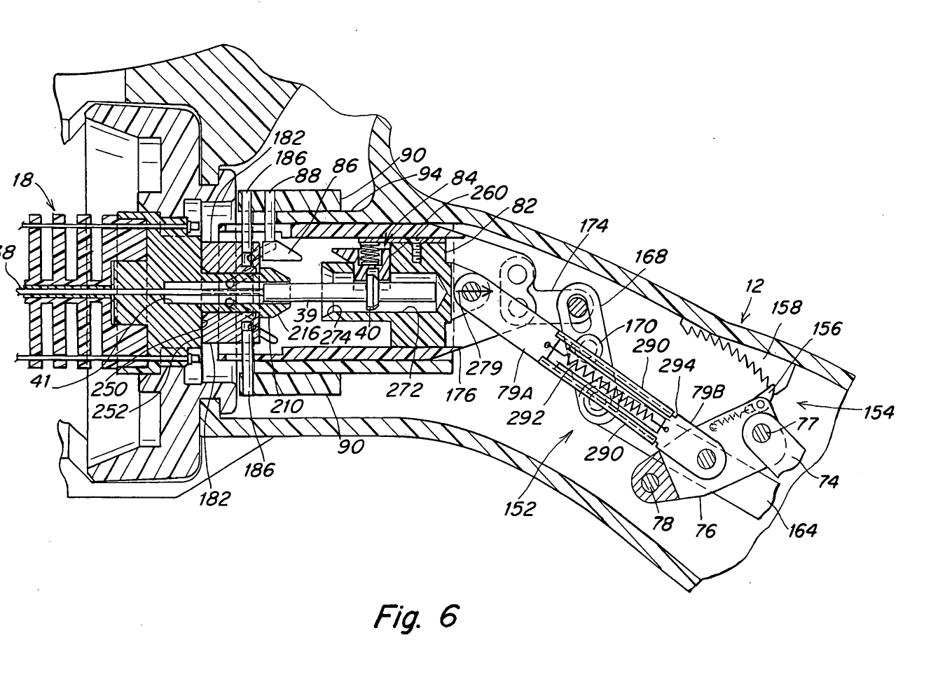

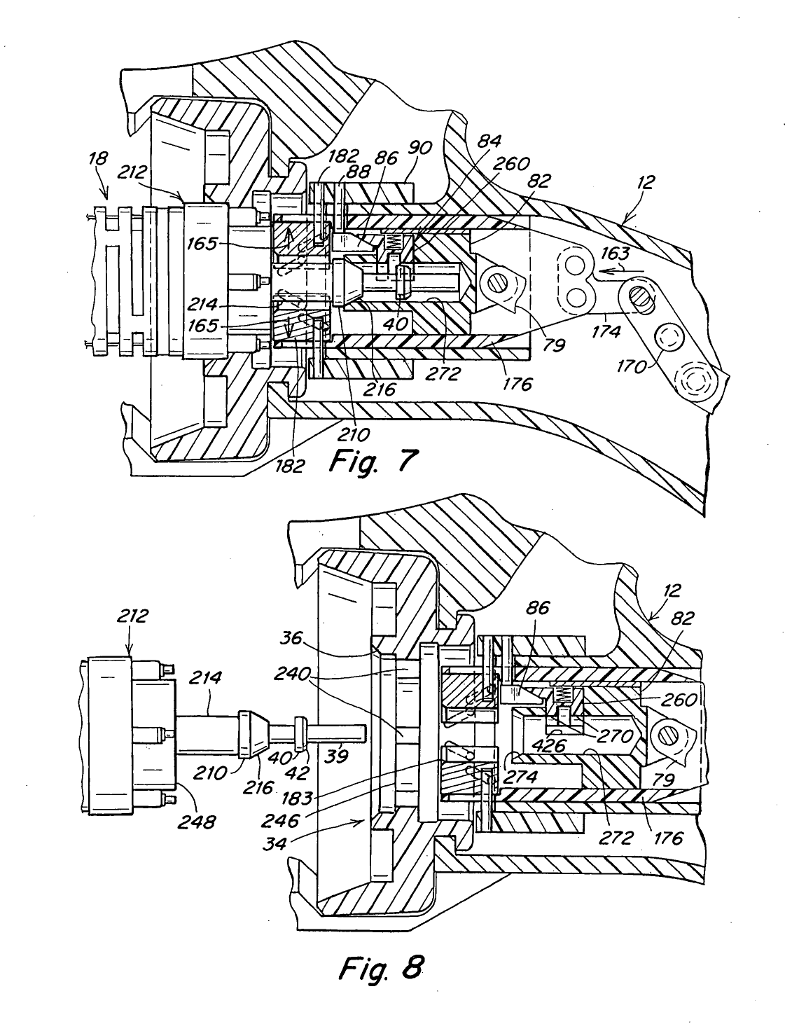

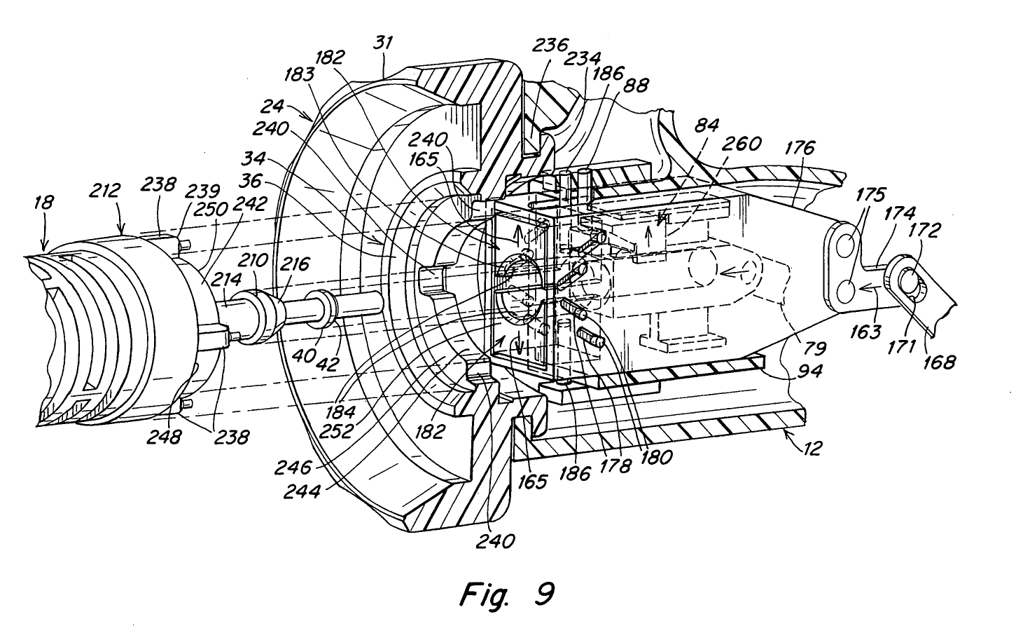

The instrument of the present invention provides the ability to re-use the handle portion of the instrument while the distal portion or shaft portion is disposable or resposable. This is enabled by providing a disconnection essentially at the proximal bendable member. As shown, for example, in FIG. 4 the shaft portion 14 includes a shaft connector 212 attached to the proximal bendable member 18. It is the shaft connector 212 that is engageable with or releasable from the receiver portion 34 of the rotation knob 24. The shaft connector 212 may be seated in the receiver portion 34 of the rotation knob and is keyed to the rotation knob 24 by means of splines 238 of the connector 212 and grooves 240 in the seat 246 of the receiver portion 34. Refer also to FIGS. 8 and 9 for further details. A reduced diameter portion 242 of the shaft connector 212 passes through a clearance hole 244 in the seat 246 of the receiver portion 34 and abuts the clamping blocks 182 (see FIG. 6) which when closed, loosely fit about a post 214 extending proximally through the semicircular bores 184. The proximal flange 210 at the end of the post 214 is relatively loosely captured by the clamping blocks allowing rotational but not axial movement of the shaft connector 212. Refer to FIGS. 7 and 9.

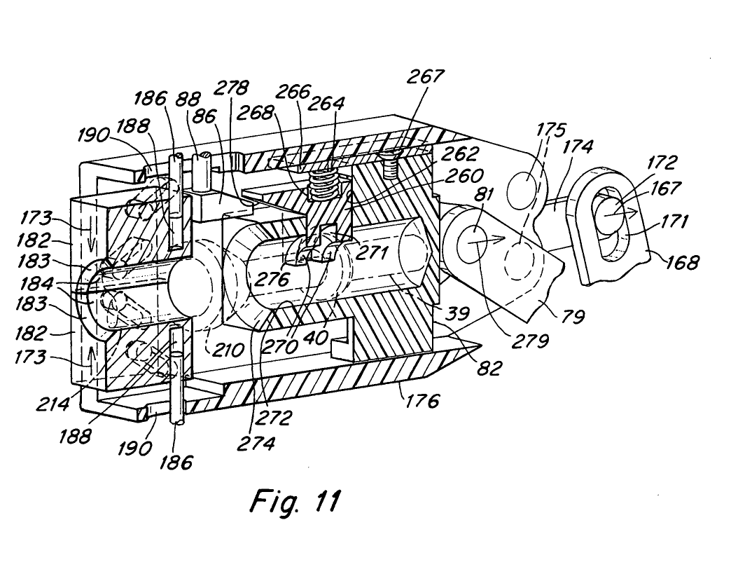

The proximal end of the push/pull cable 38 is bonded to a tube 39 that is free to slide in bore 41 of the post 214, as depicted in FIG. 5. The tube 39 maybe attached to the cable 38 in any one of a number of different ways such as by using an adhesive, soldering or crimping. The tube 39 is not illustrated as biased in any particular direction (proximally or distally), but may be spring-loaded proximally or distally to bias the jaws (or other end effector) into a desired “at rest” position. For example, a spring may be provided in the bore 41, as in FIG. 5. The tube 39 has a lug 40 that is adapted to be captured by the cable engagement means 84. The cable engagement member 84 is comprised primarily of the gate 260. The gate 260 at the handle portion is controlled from the actuation lever 22. The lug 40 has a taper 42 to aid in inserting the shaft into the handle and to provide clearance for the gate 260. The gate 260 grabs the lug 40 as the lever 22 is initially squeezed and the carriage 82 is pulled proximally as best seen in FIG. 5A where the gate 260 has its slot 271 engage the lug 40. The gate 260 moves up and down in a guide slot 262 in the carriage 82. The gate is biased to a closed position as best seen in FIG. 11 by a spring 264. The spring is retained by an arm 266 that is screwed down to the top of carriage 82. The lower end of the spring seats in a well 268 in the gate. When the gate is in the closed position the two semi-circular flanges 270 with a gap 271 between them extend into the central bore 272 in the carriage 82 to capture the cable lug 40 in the gap between the flanges.

As shown in FIG. 11, the bore 272 has a taper 274 at its distal end to guide the lug 40 into position when inserting the shaft into the handle. The gate 260 bottoms out at 276 which corresponds to the end of the guide slot 262 to allow a radial clearance between the flanges 270 and the tube 39 to allow free rotation of the lug and tube within the carriage 82. A ramp 278 on the gate 260 interacts with cam block 86 at the distal end of carriage travel to urge the gate open for release of the lug 40 when the lever 22 is at rest. This means that the cable lug 40 is normally free for shaft removal whenever the lever 22 is released or at rest, such as at the position shown in FIG. 4.

When the lever 22 is squeezed the carriage 82 is pulled proximally in the direction of arrow 279 (See FIGS. 3, 5A, 6 and 11), the ramp 278 slides down the cam block 86 and the proximal flange 270 passes over the tapered edge 42 of the lug 40 while the distal flange 270 contacts the distal face of the lug 40. This action initiates the pulling of the lug 40 in direction 279. When the stroke reaches approximately the position of FIG. 11, the ramp 278 drops off the cam block 86 and the cable lug 40 is fully captured. The further squeezing of the lever 22 toward the handle results in the operation of the ratcheting means 154. The lever 22 can then be fully squeezed to release the ratcheting member 154 and the cable engagement means 84. This action returns the carriage 82 under bias from the spring 71 until the taper 274 of the carriage nests on the taper 216 of the flange 210 which aligns the engagement means 84 with the lug 40.

The compensation means 152 as best seen in FIGS. 5 and 6 is now described. The compensation member 152 provides a bias force while at the same time accommodating different size needles or other objects at the end effector. For simplicity, the compensation means is not shown in FIGS. 1-4. The compensation means or member is comprised primarily of a link 79 that is constructed of two relative sliding portions 79a and 79B. The link 79 is supported in a guide 290 on portion 79A allowing portion 79A to be biased proximally toward portion 79A by means of the spring 292. A shoulder 294 on portion 79B acts as a stop. As shown in FIGS. 2 and 3, one end of the link 79 is supported from crank 76 at pin 80 while the opposite end is supported from carriage 82 at pin 81. Crank 76 pivots at pin 78. Link 74 is attached to crank 76 at pin 77, and intermediate pins 77 and 78. Pin 80 supports link 79 from crank 76. When the lever 22 is squeezed the jaws 44, 46 of the end effector 16 close on needle 45. After contacting the needle the link portion 79A stops movement and portion 79B continues to be pulled in a proximal direction under tension from spring 292 thus compensating for needle thickness while exerting a constant grabbing force to the jaws.

The ratchet mechanism 154 is comprised of a spring loaded pawl 156 acting in a one way ratcheting action on rack 158. The rack 158 is secured to an inner surface of the handle. In FIGS. 2 and 5 it is noted that the pawl 156 is not yet engaged with the rack 158. FIG. 3 illustrates the lever substantially depressed with the pawl 156 near the end of its travel. The pawl moves along the rack until it clears the rack which would be just past the position shown in FIG. 6. The pawl 156 is then free to pivot past the teeth of the rack 158 and thus release the crank 76 to be returned to the start position of FIG. 5 by lever return spring 71. Once the pawl passes the end of its travel it automatically returns to the position of FIG. 5 under control of the return spring 71. This action also opens the gate 260 enabling release of the more distal shaft portion.

### Shaft Portion Release

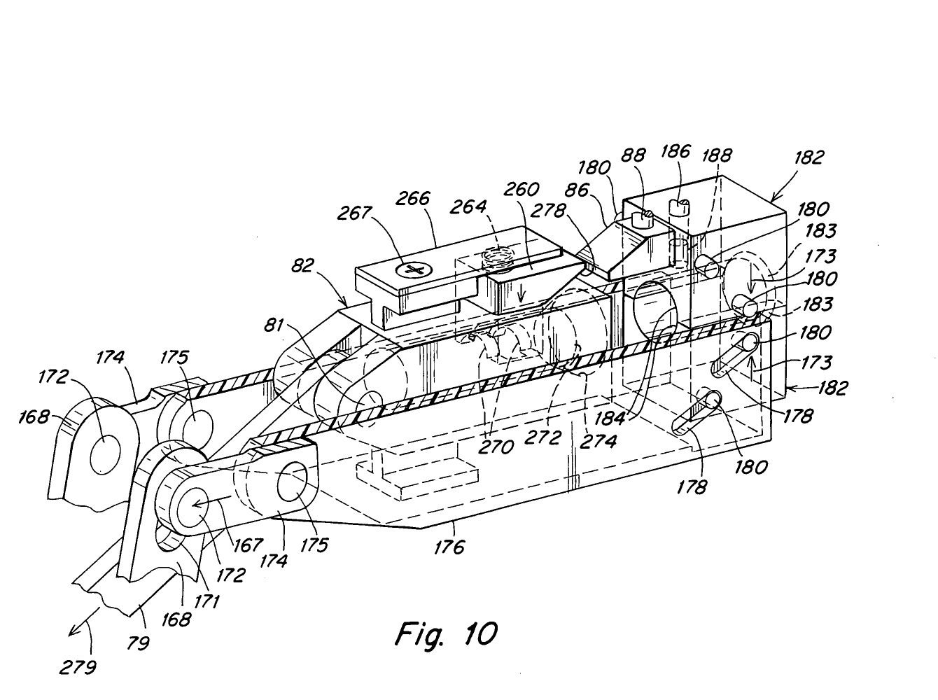

The cinch ring 200 is released so the ball 120 of the shaft portion 14 can be pulled from the split hub 202. The cinch ring is released by means of operation of the lever 220. The shaft locking means or member 150 is released by pushing the lever 160 at the base of the handle in the direction of arrow 161 as shown in FIG. 3 resulting in the pivoting of lever 160 in a clockwise direction about the pivot post 162. This action is transmitted through linkage 164 which is connected at one end to lever 160 by means of pin 166, and at the opposite end to the bell crank 168 which is connected to link 164 by pin 169 (see FIG. 5). When the lever 160 is actuated the bellcrank 168 pivots counterclockwise about pin 170 and the slot 171 in the bellcrank drives pin 172 and, in turn, brackets 174 in the distal direction of arrow 163 as shown in FIGS. 4, 5, 7 and 9. The brackets 174 (see also FIG. 10) are mounted to the rectangular sleeve 176 by screws or rivets 175. The sleeve 176 has ramped slots 178 (see FIGS. 9 and 10) that act against pins 180 mounted in clamping blocks 182. This action urges the blocks 182 apart (open) in the direction of arrows 165 as illustrated in FIGS. 7 and 9. The clamping blocks 182 are prevented from lateral movement by guide pins 186 which ride in bores 188 in the blocks. The guide pins 186 are supported on arms 90 (see FIGS. 5 and 6) which are fastened to the support tube 94 in a fixed position by means of locating pins 91 and screws 92. The guide pins 186 pass through slots 190 in sleeve 176, as illustrated in FIG. 11.

The top arm 90 also supports post 88 on which cam block 86 is mounted. The post 86 also passes through the slot 190 in the sleeve 176. The opening of the clamping blocks 182 leaves a clearance for the proximal flange 210 of the shaft connector 212 to be withdrawn through the passage created by the semi-circular bores 184 in the blocks (FIGS. 7 and 9). The shaft connector 212 can then be removed from the shaft receiver portion 34 of rotation knob 24 and, at the same time, the ball portion 120 of the shaft is pulled out of the split hub 202, as illustrated in FIGS. 4 and 7-9.

### Shaft Portion Insertion

The following description relates to the insertion sequence of the shaft portion 14. As the shaft portion is inserted, the ball 120 passes through the distal edge of split hub 202. This distal edge may be tapered as shown in FIG. 4 to assist in the insertion and to provide some guidance. The shaft connector 212 is guided into position at least by way of the taper 36 on the shaft receiver portion 34 and also by means of the tapers 239 on the splines 238 of the shaft connector 212. FIGS. 6 and 8 also show how the taper 216 of the proximal flange 210 assists in the insertion by engagement with the taper 274. Moreover, the clamping blocks 182 are each provided with a taper 183 to assist in alignment of the shaft portion 14, and as illustrated in FIG. 9. These various tapers assist in centering of the cable lug 40 as it passes into the carriage 82, such as depicted in FIG. 7.

When the ends of the splines 238 contact the seat 246 (see FIG. 9), the shaft portion 14 can then be rotated until the splines 238 align with the grooves 240. The shaft connector 212 can be inserted all the way into receiver portion 34 until the seat 246 prevents further proximal movement by contacting the shoulder 248 of the connector 212. The shoulder 250 of shaft connector 212 simultaneously contacts the face 252 of the clamping blocks 182. The shaft release lever 160 may then be pulled proximally (in a direction opposite to that of arrow 161 in FIG. 3) resulting in the proximal movement of sleeve 176 in the direction of arrow 167 in FIG. 10 which, in turn, closes the clamping blocks 182 about the post 214, capturing the annular flange 210. The arrows 173 shown in FIGS. 10 and 11 illustrate this closure and the capture of the flange 210. The release lever 160 may be provided with detents so as to keep it in either of the clamped or released positions, so that the shaft portion would not be mistakenly released. Once the shaft portion 14 is captured in the handle portion 12, then the lever 22 is used to control the actuation of the end effector. For example, FIG. 3 shows the lever at least partially depressed with the carriage 82 moved proximally and with the jaws 44, 46 closed for grasping a needle 45.

### Cautery Tool Embodiment

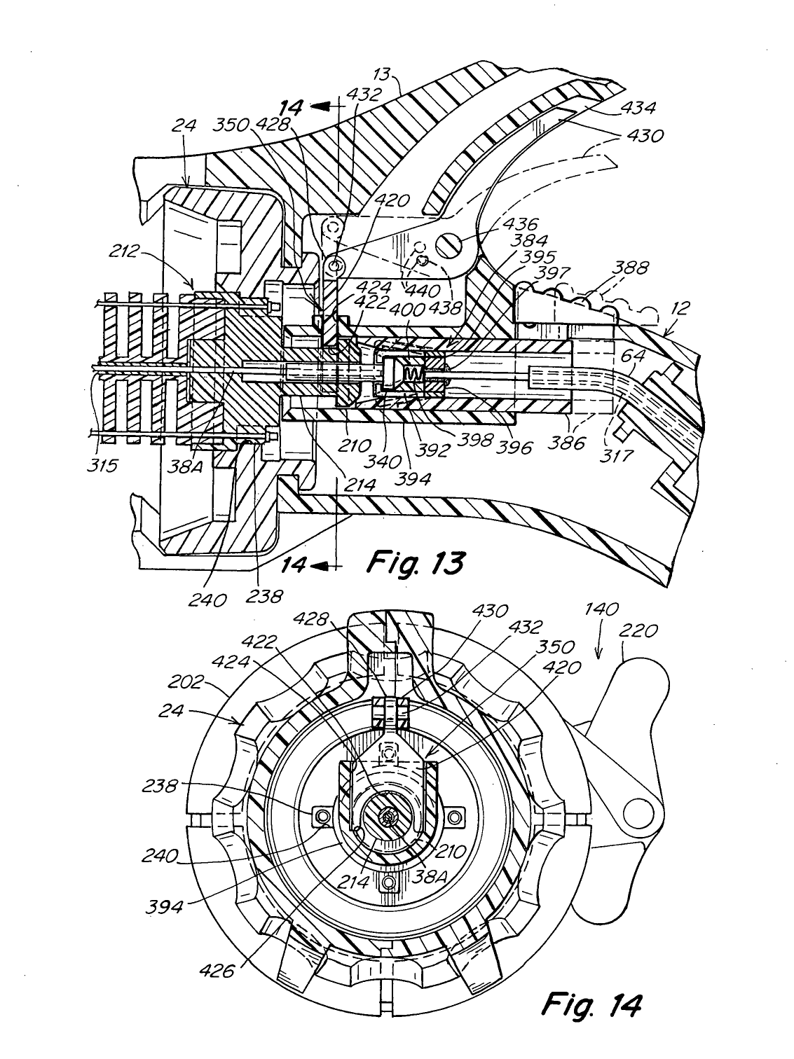

An alternate embodiment of the present invention is shown in FIG. 12 in which the instrument 310 is particularly adapted for cauterization performed in surgery. Further details are illustrated in FIGS. 13-17. This embodiment also provides for a replaceable shaft with a different release mechanism, as described hereinafter. In the previous embodiment described herein a set of jaws are depicted. In this embodiment the end effector has been replaced with a collet mechanism 316 that releasably grasps a cautery tool 320 and provides an electrical connection to the electrical contact 322 (see FIG. 12B) of the tool for enabling selective activation of the cautery tool. The cable 38 is used to clamp the collet 316 as well as provide an electrical current to heat the cautery tool. The cable is divided into two portions, one portion 38A is integral with the shaft 314 and is electrically insulated by a sheath 315 (see FIG. 12B) which also is preferably constructed of a low friction material to allow the cable to readily slide within the sheath 315. The cable portion 38B also has an insulating sheath 317 (see FIG. 13). The cable portion 38B passes through the sheath 317 and is connected at its more proximal end to a slider 28 at barrel 66.

The internal portion of the handle is not shown in detail herein but earlier applications that have been incorporated by reference herein disclose more details of the slider and barrel arrangement that may be used for actuating the cable 38. Refer, for example, to application Ser. No. 11/185,911 filed on Jul. 20, 2005; Ser. No. 11/302,654, filed on Dec. 14, 2005; Ser. No. 11/505,003 filed on Aug. 16, 2006; Ser. No. 11/528,134 filed on Sep. 27, 2006 and Ser. No. 11/649,352 filed on Jan. 2, 2007. In an alternate embodiment the barrel 66 may not be needed and the cable may be clamped directly to the slider since the cable 38A is free to rotate independently at the connector 384. The proximal end of the cable 38B then passes into a handle extension 324 that is attached to the end of the handle 12. The handle extension 324 contains a tubular electrical contact 326 that allows the cable to slide proximally and distally while maintaining electrical connection to a variable voltage source 328 that is, in turn, connected to the contact 326 at node 330 by means of the flexible cable 332. A switch (not shown) may be supported conveniently at or adjacent to the extension or variable voltage source so that the voltage can be selectively applied to the tool 320.

The collet mechanism 316 is illustrated in FIGS. 12A and 12B and is used to accept different sizes, shapes, styles, etc. of tools 320. Depending upon the particular surgical procedure, the tool is typically provided in bent configurations. In accordance with the present invention, rather than having to use different overall instruments corresponding to each type, a single instrument can be used and the different tool tips are simply replaced at the tip of the instrument in order to change tool types, sizes or shapes. The collet 360 is made of electrically insulating material such as a hard plastic and is attached to the distal end of the distal bendable member 20 and cables 100. Jaws 364 are activated to grasp and release the tool 320. In the disclosed embodiment four such jaws are used, however, it is understood that different numbers of jaws maybe employed. The base 362 of the jaws 364 contain an electrical contact 366 that may be soldered onto the distal end of cable 38A. The contact 366 mates with the contact 322 on the cautery tool. The base 362 may be constructed of metal material and it may be soldered at 368, as illustrated in FIG. 12B, to provide further electrical contact between cable 38A and the contact 322 of the cautery tool.

The cautery tool is adapted for grasping and release by the collet and jaw structure shown in FIGS. 12A and 12B. This grasping or release is controlled from the actuation cable 38. Because the cautery tool is an non-articulating tool the main cable is not needed for tool actuation and is instead used for the selective capture of the cautery tool itself. The cautery tool 320 is pushed into the relaxed jaws until contact 322 of the tool bottoms out against contact 366 in the base 362. The lever 22 may then be squeezed (depressed inwardly toward the handle) thus causing the cable 38A to pull the jaws 364 into the collet 360. This relative motion between the jaws and collet essentially closes the jaws against the tool. This is illustrated in FIG. 12B by the direction of arrows 369. The cautery tool is thus secured in the collet 360 and is electrically connected to the voltage source 328. The electrically energized jaws 364 and contact 322 are recessed from the distal end of the insulated collet 360 in order to prevent shock to the patient. The lever 22 may be provided with one or more detents so that the lever can be maintained in the particular desired position, either locked or released.

This embodiment of the invention also discloses an alternate way of engaging the shaft portion of the instrument. An alternate cable engagement means or member 284 is shown in FIGS. 13-17. This embodiment also is illustrated with a proximal bendable member 18 that has ribs defining adjacent slots as in previous instruments shown in applications incorporated herein. Many of the components in this embodiment may be the same as shown in the first embodiment herein such as the shaft connector 212, the ball 120, the rotation knob 24 and the proximal flange 210. Mainly, the alternate cable engagement member 384 is discussed in further detail herein. In the first embodiment described herein the capture of the shaft portion involved action at the release lever 160 that was located at the very proximal end of the handle. In this second embodiment separate members are used including the cable release button 388 and the release lever 430. The button 388 is for engaging the contact between the cable sections while the lever 430 is used to lock the shaft portion 314 in place relative to the handle.

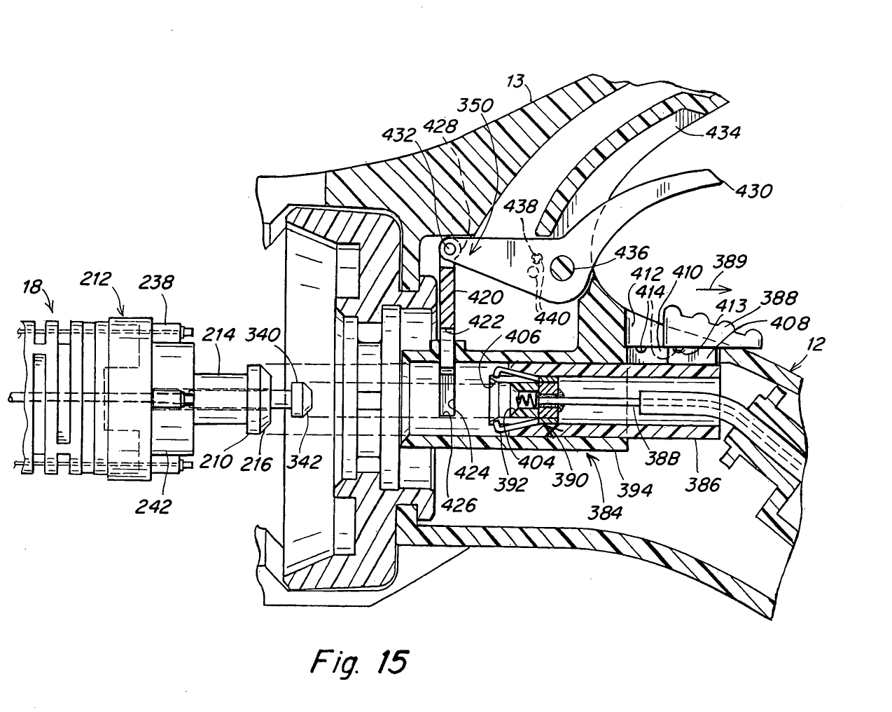

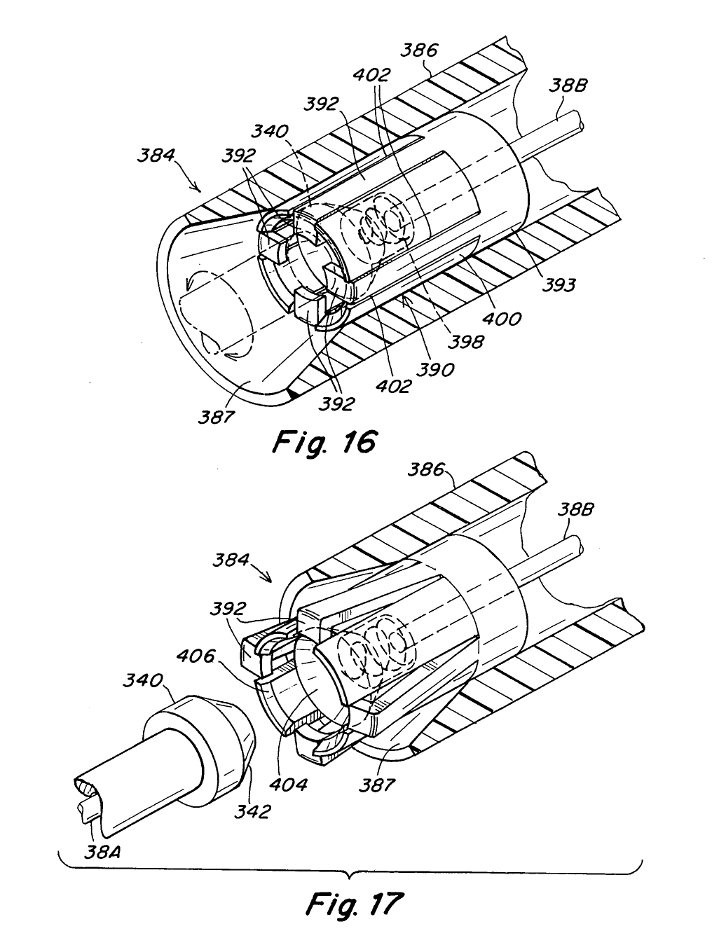

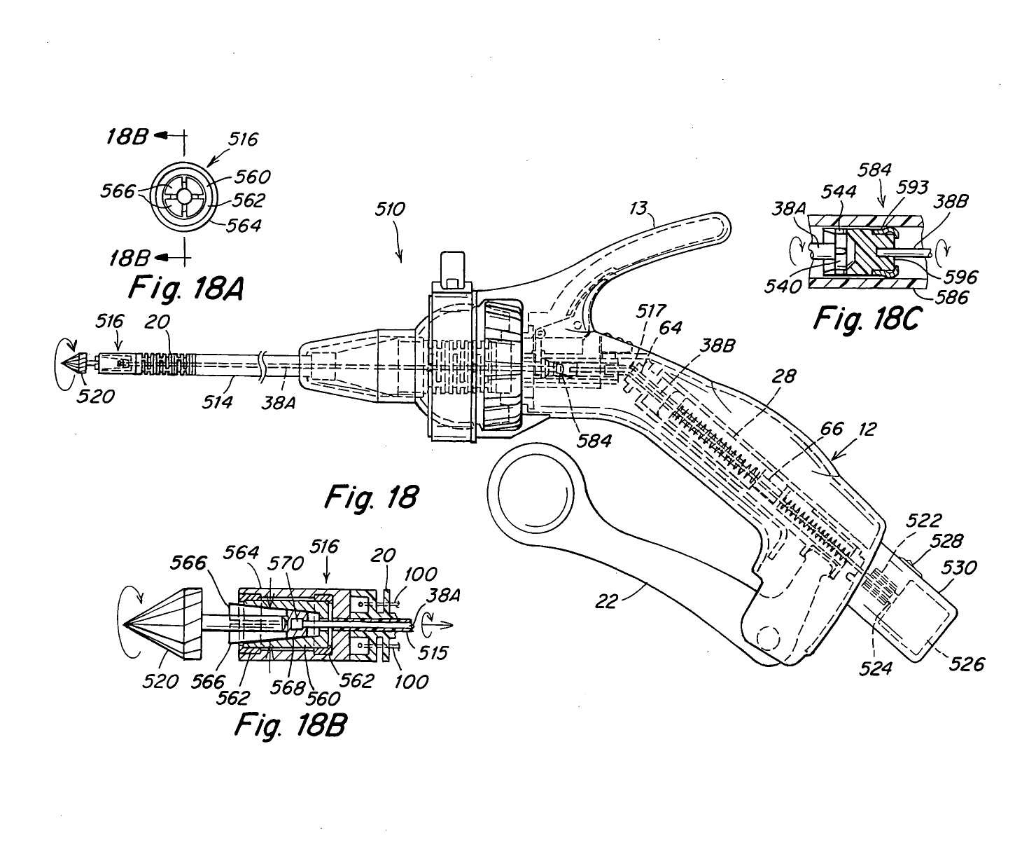

A slidable sleeve 386 is supported in the handle support tube 394 as shown in FIGS. 13 and 15. The sleeve 386 functions as a collet controlling the grasping fingers 392, and is connected to and operated from the release button 388. The sleeve 386 is slidable proximally and distally in the support tube 394 which is formed as part of the handle. The taper 387 at the distal end of the sleeve 386 (see also FIGS. 16 and 17) opens or closes fingers 392 about the lug 340. The sleeve 386 functions as a slideway for the connector 390 when the cable 38B is pulled or released by lever 22 in the process of engaging or releasing the cautery tool. The fingers 392 may be made of a metal material for electrical conduction purposes with the lug 340. The base 393 of the fingers 392 may be soldered, as illustrated at 395 in FIG. 13, to a metal core 396 that, in turn, is soldered at 397 to the bare end of the cable 38B. An electrical contact in the form of a spring 398 may be attached to the core 396 to ensure good electrical contact with the metal lug 340 which may be soldered to the cable 38A or attached in any other suitable manner. A plastic insert 400, with slots 402 for accommodating the fingers 392, includes a seat 404 (see FIG. 17) for engaging the taper 342 on the lug 340.

The insert 400 also has a taper 406 (see FIG. 17) at the distal end to aid in alignment of the lug 340 with the connector 390 when inserting the shaft into the instrument. As can be seen in FIG. 16, the cable lug 340 is free to rotate within the connector 390 but maintains electrical contact with cable 38B. The release button 388 is attached to the sleeve 386 by means of a narrow neck 408 (FIG. 15) that protrudes through a slot 410 in the handle. The release button 388 slides in and out of a recess 412 in the top of the handle just behind the horn 13. The button 388 has a nub 413 that snaps into detents 414 in the recess 412 in both locked and unlocked positions. When the button 388 is pulled in the direction 389 depicted in FIG. 15, that action pulls the sleeve 386 back from the fingers 392, letting the fingers spread open for clearance for the lug 340 to be removed or inserted. When it is in this outer or extended position the button 388 protrudes noticeably above the surface of the handle, as shown in solid outline in FIG. 15, as a clear indicator that the cable is not locked in place. If the button 388 is moved distally that action slides the sleeve 386 against the connector 390 and thus locks the cable at the fingers 392, as is depicted in solid outline in FIG. 13.

An alternate embodiment of shaft locking means is illustrated at 350, and is now described as shown in FIGS. 12-15. Instead of clamping blocks closing around the neck 214 as in the first embodiment described herein, a gate 420 with a semi-circular rim 422 (see FIG. 14) captures the flange 210 on the shaft connector 212. The gate 420 rides in a guide slot 424 formed in the support tube 394. A stop 426 (see FIG. 15) at the bottom of the slot keeps the rim 422 from contacting the post 214. A boss 428 on the top of the gate 420 is connected to release lever 430 by means of the pin 432. The lever 430 sits in a slot 434 on the underside of the horn 13 and pivots on pin 436. A nub 438 on the lever 430 snaps into detents 440 on the side of the slot 434 in both unlocked and locked positions. The lever 430 may be accessed by inserting a thumbnail at the top of the slot and pushing down. When the lever is in the unlocked position as shown in phantom line in FIG. 13 and in solid line in FIG. 15, it is a noticeable indicator that the shaft is not locked in place. The instrument is ready for use when both the button 388 and lever 430 are in their recessed positions.

### Rotary Cutting Tool Embodiment

An alternate embodiment of the surgical instrument for use as a rotary cutting tool is shown in FIG. 18. Additional details are found in FIGS. 18A, 18B, 18C, 19 and 20. The end effector 516 has a collet clamping mechanism 516 that holds a tool such as a rotary cutter 520 in this particular embodiment. It is understood that other forms of rotary tools may also be used, as well as other forms of stationary tools. The collet clamping mechanism 516 allows the collet 560 and cable portion 38A to rotate freely. The cable portion 38A passes through a low friction sheath 515 (see FIG. 18B) in the main instrument shaft and connects to cable portion 38B by means of the cable engagement means or member 584. The mechanism 584 keys the two cable portions together rotationally as well as laterally. The cable portion 38B then passes through a low friction sheath 517 (see FIG. 18) in stiffening tube 64 to the slider 28 where the stiffener tube and sheath end just short of the barrel 66. The exposed cable is then clamped to barrel 66. Barrel 66 is made of a low friction material in order to be able to rotate freely within the slider 28 when the cable 38B is driven by motor 526. The cable 38B then passes through another short section of sheath (not shown), through the end of the handle to a splined chuck 522 on motor shaft 524 of the motor 526. The cable is connected to the chuck 522. The splined chuck 522 allows limited lateral motion of the cable while transferring rotational force from the motor which can be battery driven or externally connected to a power source and controlled by a switch 528. The motor 526, switch 528 and batteries and/or external power connector are contained in housing extension 530.

The collet mechanism 516, shown in FIGS. 18A-18C, is now described. This mechanism is for locking and/or releasing the tool at the distal tip of the instrument. For this purpose a collet 560 is supported in bearings 562 within the outer housing 564 which, in turn, is connected to the distal bendable member 20 and the cables 100, as shown in FIG. 18B. Four jaws 566 clamp the tool 520 when the cable 38A, which is secured to the base 568 of the jaws by square lug 570, is pulled by squeezing the lever 22. This action pulls the tool into the collet holding the tool securely. The lever 22 may be provided with one or more detents so that the lever can be maintained in the particular desired position, either locked or released. The tool 520 which is shown as a rasp can then be rotated at high speed by the motor 526.

FIGS. 18-20 show the cable locking means or member 584 which is similar to the cable locking means 384 but modified to transmit rotational force from cable portion 38A to cable portion 38B instead of an electrical current. The connector 590 is supported and operated by a slidable sleeve 586 and taper 587 similar to the sleeve 386 and taper 387 as shown in the previous embodiment of FIGS. 12-17. Fingers 592 are supported from the base 593. These fingers 592 have slightly raised rims 594 and 595 that act as bearing surfaces against the sleeve 586. They are mainly used to reduce rotational friction as the connector spins inside the sleeve when the motor is running. They may be made of metal or plastic since they do not have to pass a current through them.

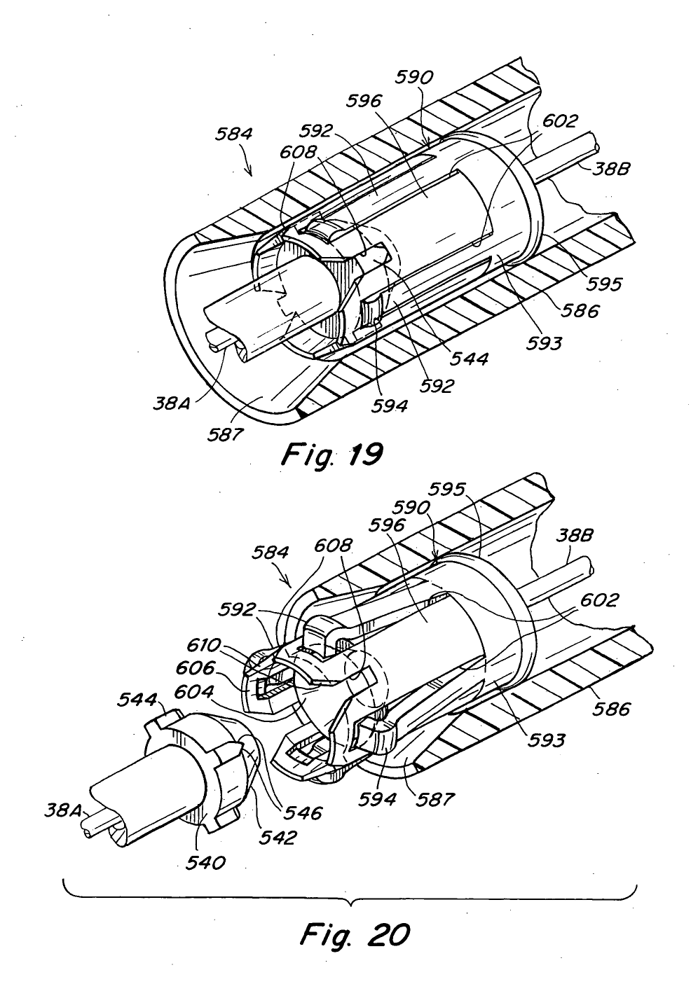

The fingers and base are mounted on a core 596 of metal or plastic that is secured to the end of cable portion 38B. The core 596 has slots 602 for the respective fingers 592 that allow the fingers to pass through and grab the lug piece 540. The core 596 has a seat 604 for receiving the lug piece 540 and a taper 606 to aid in guiding the lug upon insertion. The core has open ended slots 608 with tapers 610. The slots and tapers guide and capture the four lugs 544 with their tapers 546 that are on the circumference of each of the cable lug 544. FIG. 19 shows the fug member 540 captured with each of the lugs 544 in a corresponding slot 608 and with the fingers 592 compressed capturing the lug member 540. The rims 594 form a bearing means against the inner surface of the sleeve 586. FIG. 20, on the other hand, shows the mechanism 584 released with the fingers 592 spread and the fug member 540 out of engagement with the fingers. The mechanism 584 is partially extending out of the sleeve 586.

Having now described a limited number of embodiments relating to the principles of the present invention, it should now be apparent to one skilled in the art that numerous other embodiments and modifications thereof are contemplated as falling within the scope of the present invention, as defined by the appended claims. For example, in the first embodiment disclosed herein the cable is engaged by means of the engagement between the cable lug 40 and gate 260. In an alternate embodiment instead of a lug, a recess can be provided in the cable and instead of the slot or gap in the gate, a projection can be used for engaging with the recess. Also, the respective linkage and slider mechanisms can be interchanged between the various embodiments that are described herein.

What is claimed is:

1. A surgical instrument comprising:
an instrument shaft having proximal and distal ends;
a tool disposed from the distal end of the instrument shaft;
a control handle coupled from the proximal end of the instrument shaft;
a distal motion member for coupling the distal end of said instrument shaft to said tool;
a proximal motion member for coupling the proximal end of said instrument shaft to said handle;
actuation means extending between said distal and proximal motion members for coupling motion of said proximal motion member to said distal motion member for controlling the positioning of said tool;
said handle having a distal receiver portion;
a shaft connector on said proximal motion member selectively engageable with and releaseable from said receiver portion;
and an actuation cable extending from said shaft connector to said tool for controlling the actuation of the tool.

2. The surgical instrument of claim 1 further including a rotation means disposed adjacent the control handle and rotatable relative to the control handle for causing a corresponding rotation of the instrument shaft and tool.

3. The surgical instrument of claim 2 wherein at least said proximal motion member comprises a proximal bendable member, said rotation means comprises a rotation knob that is adapted to rotate the tool about a distal tool roll axis and said rotation knob is disposed between said control handle and proximal bendable member.

4. The surgical instrument of claim 1 including an actuation lever supported from said handle at a pivot point on the handle, a linkage mechanism controlled from said actuation lever and a cable engagement member controlled from said linkage mechanism for capturing a lug at the proximal end of said actuation cable for controlling the actuation cable and, in turn, the tool.

5. The surgical instrument of claim 4 wherein said linkage mechanism includes a ratchet and pawl mechanism that provides successive lever positions for controlling the force applied at the tool and a plurality of connected links one of which includes a split link having a biasing spring therebetween.

6. The surgical instrument of claim 4 wherein the cable engagement member comprises a carriage that supports a gate that is movable transverse to the longitudinal axis of the carriage so as to capture the cable lug.

7. The surgical instrument of claim 6 including a spring for biasing the gate and a cam block that is engageable with the gate to open the gate to enable the cable lug to be released.

8. The surgical instrument of claim 1 including a locking mechanism for fixing the position of the tool at a selected position and having locked and unlocked states, said locking mechanism including a ball and socket arrangement disposed about said proximal motion member and a cinch member for locking said ball and socket arrangement.

9. The surgical instrument of claim 8 wherein the socket member comprises a split socket and said cinch member closes said split socket to lock the socket on the ball.

10. The surgical instrument of claim 1 including a set of clamping blocks, said cable having distal of said lug a flange that is captured by said clamping blocks, said clamping blocks operated from a release member at the proximal end of the handle.

11. The surgical instrument of claim 10 including a sleeve member, a linkage member for controlling the transition of said sleeve member from said release member, said sleeve member controlling the clamping blocks to move toward and away from each other in providing the clamping action at the cable flange.

12. The surgical instrument of claim 1 including an actuation lever supported from said handle at a pivot point on the handle, said actuation cable having separable proximal and distal cable portions, said proximal cable portion controlled from said actuation lever, said distal cable portion selectively engageable or releaseable with respect to said proximal cable portion.

13. The surgical instrument of claim 12 including a cable locking mechanism for engaging the cable portions and a shaft locking mechanism for retaining the shaft connector.

14. The surgical instrument of claim 13 wherein the cable locking mechanism includes a sleeve and a release button mounted on the handle, and a connector at the distal end of the proximal cable portion that has multiple fingers for selective engagement with a lug on the distal cable portion so as to capture the cable lug, and wherein the shaft locking mechanism includes a gate and a release lever mounted on the handle, said gate for capturing a post on the distal cable portion.

15. The surgical instrument of claim 1 wherein said tool includes a collet, a removable tool member that is received in the collet and a set of jaws for holding the tool member.

16. The surgical instrument of claim 15 wherein the tool member is a cautery tool and further including a voltage source at the handle for coupling energy to the actuation cable and an actuation lever for controlling the actuation cable which, in turn, controls said set of jaws for grasping the tool member.

17. The surgical instrument of claim 15 wherein the tool comprises a rotary cutter and further including a motor on the handle for controlling the rotary cutter via the actuation cable, and an actuation lever for controlling the actuation cable.

18. In a medical instrument having a proximal control handle and a distal tool that are intercoupled by an elongated instrument shaft that is meant to pass internally of an anatomic body, proximal and distal movable members that respectively intercouple said proximal control handle and said distal tool with said instrument shaft, cable control means disposed between said movable members, an actuation member at said handle for controlling said distal tool through said movable members, a tool coupler for selectively engaging or disengaging said distal tool and a control member mounted at said handle for controlling said tool coupler.

19. The medical instrument of claim 18 wherein said tool coupler includes a collet and a jaw member that transitions relative to the collet for receiving the distal tool.

20. The medical instrument of claim 19 wherein the collet is attached to the proximal movable member, the proximal movable member comprises a proximal bendable member and the jaw member comprises a set of fingers extending from a base.

21. A method of controlling a medical instrument that has a proximal end including a control handle and a distal end including a distal tool, said control handle and distal tool being intercoupled by an elongated instrument shaft and said tool actuated from a tool control cable that is operated from an actuation lever at the handle, said method including providing proximal and distal movable members that respectively intercouple said proximal control handle and said distal tool with said instrument shaft, said proximal and distal movable members being intercoupled so that a motion at said proximal movable member controls said distal movable member, and supporting the proximal movable member for removable interlock with a receiver portion at the handle.

22. The method of claim 21 including dividing the tool control cable into separate cable segments and interlocking the separate cable segments so that the tool control cable is operable.

23. The method of claim 21 including manually controlling, from the proximal end of the instrument, the rotation of said distal tool about its longitudinal distal tool axis.

24. An instrument having a proximal control handle and a distal tool that are intercoupled by an elongated instrument shaft, proximal and distal movable members that respectively intercouple said proximal control handle and said distal tool with said instrument shaft, means disposed between said movable members so that a motion at said proximal movable member controls said distal movable member and, in turn, the distal tool, means supported at the handle for controlling the distal tool including a tool control cable that extends between the proximal movable member and the distal tool and an actuation lever mounted at the handle, said handle having a distal receiver portion, and a shaft connector on said proximal movable member selectively engageable with and releaseable from said receiver portion.

25. The instrument of claim 24 wherein said tool control cable including separate control cable segments that are adapted to have one of an engaged state and a dis-engaged state.

26. The instrument of claim 24 including a control member at said control handle and manipulable by a user to control, via said proximal and distal movable members, the rotation of said distal tool about its distal tool axis.
